(19) United States
(12) Patent Application Publication
(10) Pub. No.: US 2023/0329586 A1
(43) Pub. Date: Oct. 19, 2023

(54) GAIT INFORMATION GENERATION DEVICE, GAIT INFORMATION GENERATION METHOD, AND RECORDING MEDIUM

(71) Applicant: NEC Corporation, Tokyo (JP)

(72) Inventors: Zhenwei WANG, Tokyo (JP); Kenichiro Fukushi, Tokyo (JP); Kazuki Ihara, Tokyo (JP); Kentaro Nakahara, Tokyo (JP); Fumiyuki Nihey, Tokyo (JP); Hiroshi Kajitani, Tokyo (JP); Chenhui Huang, Tokyo (JP); Yoshitaka Nozaki, Tokyo (JP)

(73) Assignee: NEC Corporation, Tokyo (JP)

(21) Appl. No.: 18/129,387

(22) Filed: Mar. 31, 2023

(30) Foreign Application Priority Data
Apr. 19, 2022 (JP) 2022-068759

(51) Int. Cl. A61B 5/11; A61B 5/00

(52) U.S. Cl. CPC A61B 5/112; A61B 5/1128; A61B 5/1114; A61B 5/742

(57) ABSTRACT

Provided is a gait information generation device including an acquisition unit that acquires waist position information including time-series data of a waist position of a subject in a predetermined gait cycle, a waist oscillation calculation unit that calculates, for a plurality of gait phases included in the predetermined gait cycle, a waist oscillation corresponding to a distance between a reference line set for the time-series data of the waist position of the subject and the waist position in each of the plurality of gait phases, a gait information generation unit that generates gait information according to the waist oscillation calculated with respect to the predetermined gait cycle, and an output unit that outputs the generated gait information.

This application is based upon and claims the benefit of priority from Japanese Patent Application No. 2022-068759, filed on Apr. 19, 2022, the disclosure of which is incorporated herein in its entirety by reference.

### TECHNICAL FIELD

The present disclosure relates to a gait information generation device or the like that generates information according to a feature included in a gait pattern.

### BACKGROUND ART

With increasing interest in healthcare, services for providing information (also referred to as gait information) related to features (also referred to as gait) included in a gait pattern have attracted attention. When the gait information with which the gait can be intuitively grasped can be presented, the healthcare service according to the user's needs can be provided.

Patent Literature 1 (JP 2016-034478 A) discloses a motion analysis method for analyzing motion information about a user. In the method of Patent Literature 1, the motion of the user is analyzed using the detection result of the inertial sensor. In the method of Patent Literature 1, a plurality of pieces of motion information is generated for a user who is exercising. In the method of Patent Literature 1, a comparison result between at least one piece of motion information among a plurality of pieces of motion information and a preset reference value is presented to the user.

Patent Literature 2 (WO 2020/105115 A) discloses a gait measurement system that calculates a gait index based on acceleration data measured by an inertial measurement unit. The system of Patent Literature 2 detects at least one gait phase from acceleration data measured by an inertial measurement unit. The system of Patent Literature 2 calculates speed data by time-integrating acceleration data. The system of Patent Literature 2 calculates a correction amount related to the gait phase using the gait phase and the speed data. The system of Patent Literature 2 calculates corrected speed data by subtracting the correction amount from the speed data related to the gait phase, and calculates locus data by time-integrating the calculated corrected speed data. The system of Patent Literature 2 calculates a gait index that is a numerical value for quantitatively evaluating the gait using the calculated locus data.

According to the method of Patent Literature 1, a comparison result between the motion information about the user and the reference value can be presented. However, in the method of Patent Literature 1, it is not possible to present information with which the user's motion can be intuitively grasped.

According to the method of Patent Literature 2, since the gait index is calculated using the locus data obtained by time-integrating the corrected speed data corrected in association with the gait phase, the gait can be accurately measured. However, in the method of Patent Literature 2, it is not possible to present information with which the gait can be intuitively grasped.

An object of the present disclosure is to provide a gait information generation device and the like that generate gait information that enables intuitive grasp of a gait of a subject.

### SUMMARY

A gait information generation device according to an aspect of the present disclosure includes an acquisition unit that acquires waist position information including time-series data of a waist position of a subject in a predetermined gait cycle, a waist oscillation calculation unit that calculates, for a plurality of gait phases included in the predetermined gait cycle, a waist oscillation corresponding to a distance between a reference line set for the time-series data of the waist position of the subject and the waist position in each of the plurality of gait phases, a gait information generation unit that generates gait information according to the waist oscillation calculated with respect to the predetermined gait cycle, and an output unit that outputs the generated gait information.

A gait information generation method according to an aspect of the present disclosure includes acquiring waist position information including time-series data of a waist position of a subject in a predetermined gait cycle, calculating, for a plurality of gait phases included in the predetermined gait cycle, waist oscillation corresponding to a distance between a reference line set for the time-series data of the waist position of the subject and the waist position in each of the plurality of gait phases, generating gait information according to the waist oscillation calculated with respect to the predetermined gait cycle, and outputting the generated gait information.

A recording medium according to an aspect of the present disclosure records a program for causing a computer to execute the steps of acquiring waist position information including time-series data of a waist position of a subject in a predetermined gait cycle, calculating, for a plurality of gait phases included in the predetermined gait cycle, waist oscillation corresponding to a distance between a reference line set for the time-series data of the waist position of the subject and the waist position in each of the plurality of gait phases, generating gait information according to the waist oscillation calculated with respect to the predetermined gait cycle, and outputting the generated gait information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a gait information generation device according to a first example embodiment;

FIG. 2 is a conceptual diagram for describing an example of a gait event related to gait information generated by the gait information generation device according to the first example embodiment;

FIG. 3 is a conceptual diagram illustrating an example of a relationship between a following center of a virtual camera and a waist position in display information included in gait information generated by the gait information generation device according to the first example embodiment;

FIG. 4 is a conceptual diagram illustrating a display example of display information included in the gait information generated by the gait information generation device according to the first example embodiment;

FIG. 5 is a graph illustrating an example of time-series data of a waist position used for calculating the waist position by the gait information generation device according to the first example embodiment;

FIG. 6 is a conceptual diagram for describing calculation of a waist position by the gait information generation device according to the first example embodiment;

FIG. 7 is a graph illustrating an example of time-series data of a waist position calculated by the gait information generation device according to the first example embodiment;

FIG. 8 is a conceptual diagram illustrating a display example of display information generated by the gait information generation device according to the first example embodiment;

FIG. 9 is a graph illustrating an example of time-series data of a waist position used for calculating a waist position over a plurality of gait cycles by the gait information generation device according to the first example embodiment;

FIG. 10 is a graph illustrating an example of time-series data of a waist position over a plurality of gait cycles calculated by the gait information generation device according to the first example embodiment;

FIG. 11 is a flowchart for explaining an example of an operation of the gait information generation device according to the first example embodiment;

FIG. 12 is a conceptual diagram for explaining Application Example 1-1 according to the first example embodiment;

FIG. 13 is a conceptual diagram for explaining Application Example 1-2 according to the first example embodiment;

FIG. 14 is a conceptual diagram for explaining Application Example 1-3 according to the first example embodiment;

FIG. 15 is a conceptual diagram for explaining Application Example 1-3 according to the first example embodiment;

FIG. 16 is a block diagram illustrating an example of a configuration of a gait information generation device according to a second example embodiment;

FIG. 17 is a graph for describing calculation of a speed of the waist oscillation by the gait information generation device according to the second example embodiment;

FIG. 18 is a conceptual diagram illustrating a display example of display information generated by the gait information generation device according to the second example embodiment;

FIG. 19 is a flowchart for explaining an example of an operation of the gait information generation device according to the second example embodiment;

FIG. 20 is a conceptual diagram for explaining Application Example 2-1 according to the second example embodiment.

FIG. 21 is a block diagram illustrating an example of a configuration of a gait information generation device according to a third example embodiment;

FIG. 22 is a conceptual diagram illustrating an example of a relationship between a following center and a waist position in display information included in gait information generated by the gait information generation device according to the third example embodiment;

FIG. 23 is a conceptual diagram illustrating a display example of display information included in gait information generated by the gait information generation device according to the third example embodiment;

FIG. 24 is a flowchart for explaining an example of an operation of the gait information generation device according to the third example embodiment;

FIG. 25 is a conceptual diagram for explaining Application Example 3-1 according to the third example embodiment;

FIG. 26 is a conceptual diagram for explaining Application Example 3-2 according to the third example embodiment;

FIG. 27 is a block diagram illustrating an example of a configuration of a gait information generation device according to a fourth example embodiment; and

FIG. 28 is a block diagram illustrating an example of a hardware configuration that executes processing according to each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, a configuration of a gait information generation device according to the first example embodiment will be described with reference to the drawings. The gait information generation device according to the present example embodiment calculates the fluctuation of the waist (also referred to as waist oscillation) using waist position information in the traveling direction measured according to the gait of a subject to be verified (also referred to as a user). The gait information generation device according to the present example embodiment generates information (also referred to as gait information) according to a feature (also referred to as a gait) included in the gait pattern according to the calculated waist oscillation.

(Configuration)

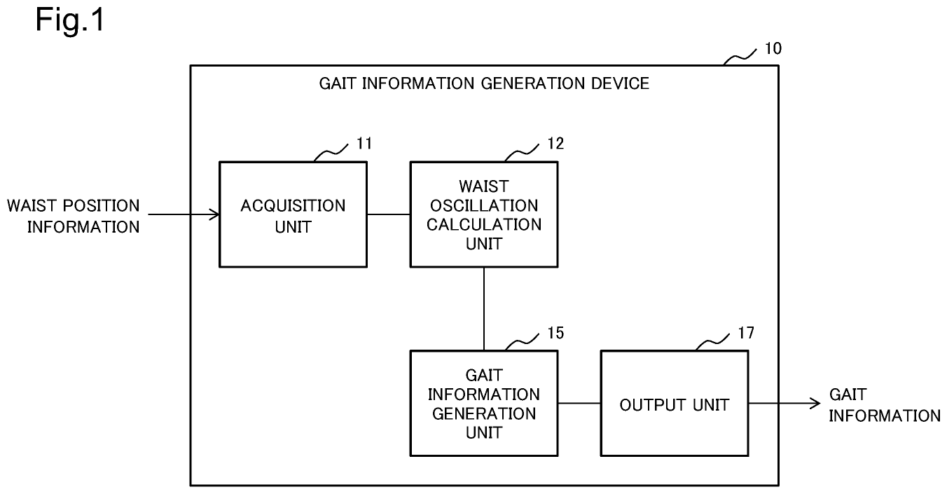

FIG. 1 is a block diagram illustrating a configuration of a gait information generation device 10 according to the present example embodiment. The gait information generation device 10 includes an acquisition unit 11, a waist oscillation calculation unit 12, a gait information generation unit 15, and an output unit 17.

Acquisition unit 11 acquires waist position information about the subject in the traveling direction. Acquisition unit 11 acquires waist position information in the predetermined gait section. For example, the predetermined gait section is one gait cycle. The predetermined gait section may be a plurality of gait cycles. In the following description, a period from landing of the heel of the right foot to landing of the heel of the right foot again is defined as one gait cycle.

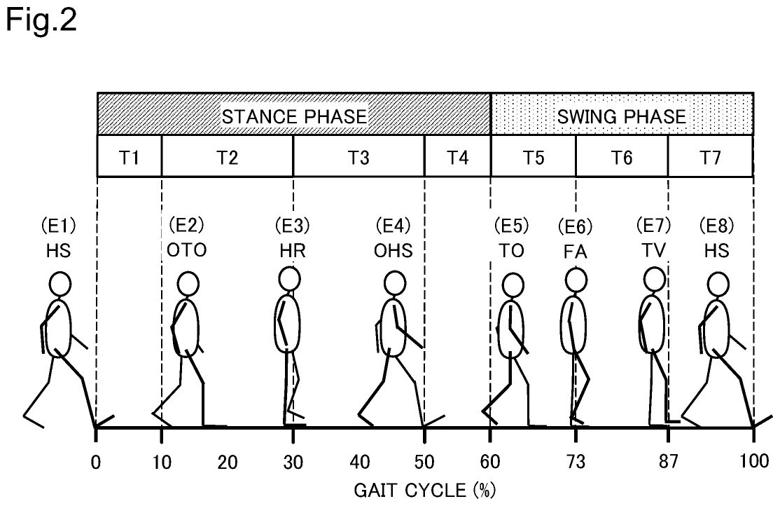

FIG. 2 is a conceptual diagram for explaining a gait event detected in one gait cycle with the right foot as a reference. The horizontal axis of FIG. 2 is a gait cycle normalized with one gait cycle of the right foot as 100% (%) with a time point at which the heel of the right foot lands on the ground as a starting point and a time point at which the heel of the right foot next lands on the ground as an ending point. Each of the plurality of timings included in one gait cycle is referred to as a gait phase. The one gait cycle of one foot is roughly divided into a stance phase in which at least part of the back side of the foot is in contact with the ground and a swing phase in which the back side of the foot is away from the ground. In the example of FIG. 2, the gait cycle is normalized such that the stance phase occupies 60% and the swing phase occupies 40%. The stance phase is further subdivided into a loading response period T1, a mid-stance period T2, a terminal stance period T3, and a pre-swing period T4. The swing phase is further subdivided into an initial swing period T5, a mid-swing period T6, and a terminal swing period T7. The gait waveform in one gait cycle may not start from the time point when the heel lands on the ground as a starting point. For example, the starting point of the gait waveform in one gait cycle may be set at the central point of time of the stance phase.

A gait event E1 represents the heel strike (HS) at the beginning of one gait cycle. The heel strike is an event in which the heel of the right foot, which has been away from the ground in the swing phase, lands on the ground. A gait event E2 represents an opposite toe off (OTO). The opposite toe off is an event in which the toe of the left foot is away from the ground in a state where the ground contact surface of the sole of the right foot is in contact with the ground. A gait event E3 represents a heel rise (HR). The heel rise is an event in which the heel of the right foot is lifted while the ground contact surface of the sole of the right foot is in contact with the ground. A gait event E4 represents an opposite heel strike (OHS). The opposite heel strike is an event in which the heel of the left foot, which has been away from the ground in the swing phase of the left foot, lands on the ground. A gait event E5 represents a toe off (TO). The toe off is an event in which the toe of the right foot is away from the ground in a state where the ground contact surface of the sole of the left foot is in contact with the ground. A gait event E6 represents a foot adjacent (FA). The foot adjacent is an event in which the left foot and the right foot cross each other in a state where the ground contact surface of the sole of the left foot is in contact with the ground. A gait event E7 represents a tibia vertical (TV). The tibia vertical is an event in which the tibia of the right foot is substantially perpendicular to the ground while the sole of the left foot is in contact with the ground. A gait event E8 represents a heel strike (HS) at the end of one gait cycle. The gait event E8 corresponds to the ending point of the gait cycle starting from the gait event E1 and corresponds to the starting point of the next gait cycle.

The waist position information is a temporal change in information about the position of the waist in the traveling direction. In the present example embodiment, the front in the traveling direction is defined as positive, and the rear in the traveling direction is defined as negative. A method of measuring the waist position information is not particularly limited.

For example, the waist position information is measured by motion capture. In motion capture, a marker is attached to each part of the subject's body. For example, the marker is attached to a site including the waist. A gait subject is photographed with a camera, and the position of the marker in the photographed image (video) is measured. According to the motion capture, since the position of the waist can be directly measured, highly accurate waist position information can be obtained.

For example, the waist position information is measured by analyzing an image (video) captured by the camera. By using software such as OpenPose, the waist position information is measured by calculating the position of the waist based on the position of the skeleton or the joint detected from the person in the image.

For example, the waist position information is measured using acceleration or angular velocity measured by an inertial sensor attached to the waist. When the inertial sensor is used, the position of the waist can be calculated by integrating the acceleration and the angular velocity. For example, the waist position information may be measured using a smart apparel in which an inertial sensor is attached to each part of the entire body.

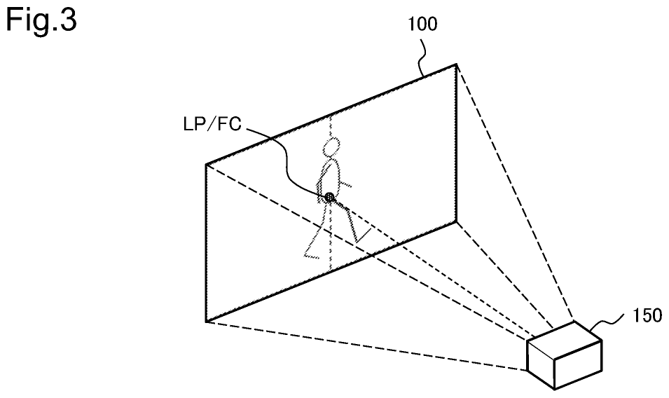

FIG. 3 is a conceptual diagram illustrating an example in which a gait subject is displayed on a screen 100. The subject is displayed at the center of the screen 100. A viewpoint (following center FC) of a virtual camera 150 that follows the subject is set at a position that captures the center of the screen 100. A waist position LP of the subject is fixed to the center of the screen 100. In the example of FIG. 3, the position of the following center FC coincides with the position of the waist position LP.

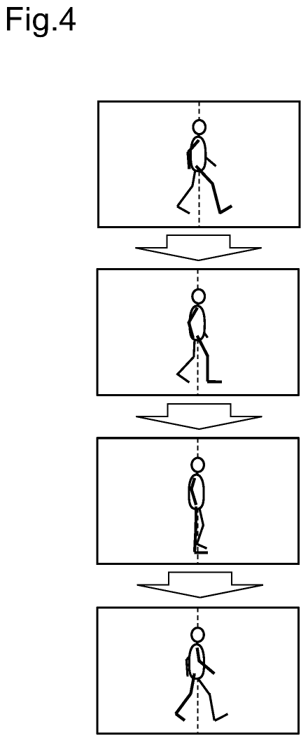

FIG. 4 is a conceptual diagram illustrating an example of a change in an image according to gait of a subject followed by the virtual camera 150 installed as illustrated in FIG. 3. A viewpoint (following center FC) of the virtual camera 150 is fixed toward the center of the screen 100. Therefore, the waist position LP of the subject hardly fluctuates, and the relative movement of the foot and hand with respect to the waist position LP is displayed.

The waist oscillation calculation unit 12 acquires the waist position information about the subject from the acquisition unit 11. Using the acquired waist position information, the waist oscillation calculation unit 12 calculates a position difference (also referred to as waist oscillation) between the position of the subject and the waist position according to the average speed. The waist oscillation is a position difference related to the movement of the waist in the predetermined gait section. The waist oscillation calculation unit 12 calculates the waist oscillation based on the gait speed in the predetermined gait section. For example, assuming that the gait speed in the predetermined gait section is the uniform linear motion, the waist oscillation calculation unit 12 approximates the gait speed in the predetermined gait section with a straight line (also referred to as a reference straight line). For each of the plurality of gait phases included in the predetermined gait section, the waist oscillation calculation unit 12 calculates the distance between the waist position and the reference straight line as the waist oscillation of the gait phase.

The waist oscillation calculation unit 12 may calculate the waist oscillation by approximating the gait speed in the predetermined gait section with a curve. For example, the waist oscillation calculation unit 12 approximates the gait speed in the predetermined gait section with a curve (also referred to as a reference curve). For example, the waist oscillation calculation unit 12 sets a reference curve that smoothly connects the starting point and the ending point with respect to the time-series data of the waist position in the predetermined gait section. For example, the waist oscillation calculation unit 12 sets a Bezier curve, a spline curve, or the like that smoothly connects the starting point and the ending point as the reference curve. For each of the plurality of gait phases included in the predetermined gait section, the waist oscillation calculation unit 12 calculates the distance between the waist position and the reference curve as the waist oscillation of the gait phase. The reference straight line and the reference curve are collectively referred to as reference lines.

The waist oscillation calculation unit 12 may calculate the waist oscillation by dividing the section into a plurality of sections cut out from the time-series data of the waist position in one gait cycle. For example, the waist oscillation calculation unit 12 sets the reference straight line and the reference curve for each of the plurality of sections. The waist oscillation calculation unit 12 calculates a distance between the reference straight line or the reference curve set for each of the plurality of sections and the waist position for each gait phase as the waist oscillation of the gait phase.

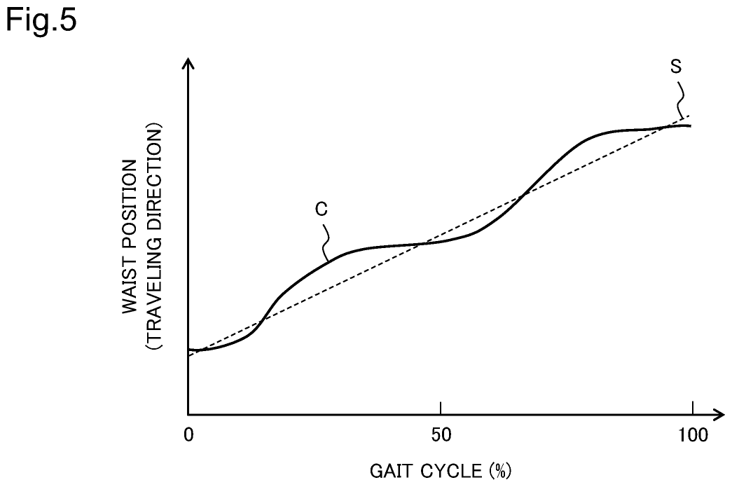

FIG. 5 is a graph for describing an example of time-series data of a waist position in a traveling direction. The graph of FIG. 5 illustrates time-series data C of the waist position in the traveling direction in one gait cycle. The waist position changes at different speeds according to the gait cycle. FIG. 5 illustrates a reference straight line S obtained by approximating the time-series data C of the waist position in one gait cycle by a linear function. For example, the reference straight line S is a regression straight line of the time-series data C of the waist position in one gait cycle. The waist oscillation calculation unit 12 calculates a distance between each point of the time-series data C of the waist position in one gait cycle and the reference straight line S as the waist oscillation. The waist oscillation is an index of the waist position based on the position of the subject according to the average speed. When the waist oscillation is positive, the waist position is located ahead of the position of the subject according to the average speed. When the waist oscillation is negative, the waist position is located behind the position of the subject according to the average speed. When the waist oscillation is used, the movement of the subject that cannot be grasped only by the movement of the foot can be grasped.

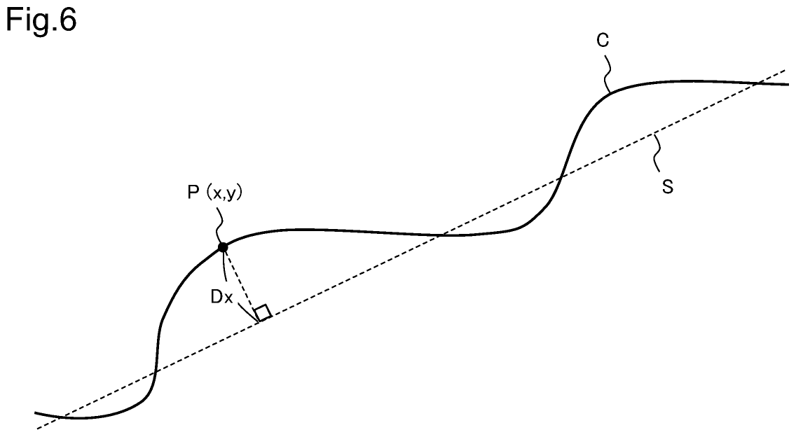

FIG. 6 is a conceptual diagram illustrating the waist oscillation in the graph of FIG. 5 in an emphasized manner. A point P(x, y) of the time-series data C of the waist position indicates the waist position in a gait phase x. The length of the perpendicular drawn from the point P(x, y) of the time-series data C of the waist position to the reference straight line S is a waist oscillation Dx in the gait phase x. When the waist position of the subject is ahead of the position of the subject according to the average speed in the predetermined gait section, the waist oscillation Dx is positive. When the waist position of the subject is behind the position of the subject according to the average speed in the predetermined gait section, the waist oscillation Dx is negative. The farther the waist position is from the position of the subject according to the average speed, the larger the absolute value of the waist oscillation Dx is. The closer the waist position is to the position of the subject according to the average speed, the smaller the absolute value of the waist oscillation Dx is.

The gait information generation unit 15 acquires the waist oscillation calculated by the waist oscillation calculation unit 12. The gait information generation unit 15 generates the gait information according to the waist oscillation calculated by the waist oscillation calculation unit 12. For example, the gait information generation unit 15 generates time-series data of the waist oscillation as the gait information. For example, the gait information generation unit 15 generates a graph indicating time-series data of the waist oscillation as the gait information. The gait information generated by the gait information generation unit 15 is not limited to the time-series data or the graph of the waist oscillation.

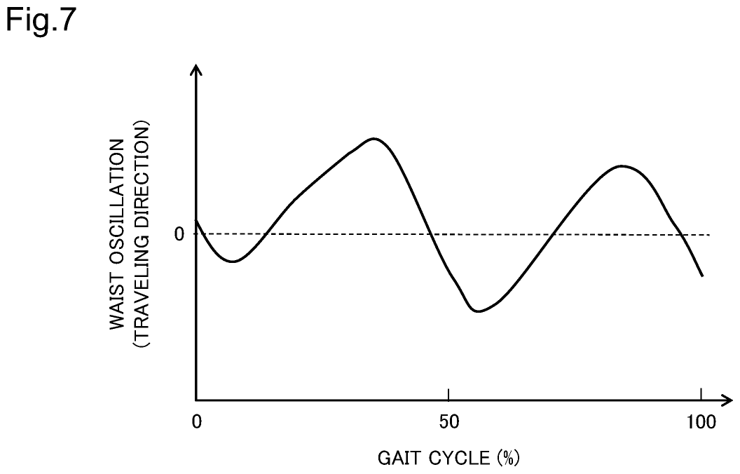

FIG. 7 is a graph illustrating an example of the gait information generated by the gait information generation unit 15. FIG. 7 is an example in which the time-series data of the waist oscillation is displayed in association with the gait cycle. According to the graph of FIG. 7, it is possible to intuitively grasp the fluctuation of the waist oscillation in accordance with the gait cycle. That is, according to the graph of FIG. 7, the movement of the subject during gait that cannot be grasped only by the movement of the foot can be grasped.

The output unit 17 outputs the gait information generated by the gait information generation unit 15. For example, the output unit 17 outputs the gait information to a terminal device having a screen. The gait information output to the terminal device is displayed on a screen of the terminal device. For example, the output unit 17 displays the gait information on a screen of a mobile terminal of the subject (user). For example, the output unit 17 displays the gait information on a screen of a terminal device used by an expert such as a doctor, a physical therapist, or a care worker who verifies the physical condition of the subject. The expert can give diagnosis of and advice to the subject according to the gait information displayed on the screen of the terminal device to the subject. For example, the output unit 17 may output the gait information to an external system or the like that uses the gait information. The use of the gait information output from the output unit 17 is not particularly limited.

For example, the gait information generation device 10 is connected to an external system or the like constructed in a cloud or a server via a mobile terminal (not illustrated) carried by a subject (user). The mobile terminal is a portable communication device. For example, the mobile terminal is a portable communication device having a communication function, such as a smartphone, a smart watch, or a mobile phone.

For example, the gait information generation device 10 is connected to a terminal device (not illustrated) used by a person who verifies the physical condition of the subject (user). Software for processing the gait information and displaying an image according to the gait information is installed in the terminal device. For example, the terminal device is an information processing apparatus such as a stationary personal computer, a notebook personal computer, a tablet, or a mobile terminal. The terminal device may be a dedicated terminal that processes the gait information.

For example, the gait information generation device 10 is connected to a mobile terminal or a terminal device via a wire such as a cable. For example, the gait information generation device 10 is connected to a mobile terminal or a terminal device via wireless communication. For example, the gait information generation device 10 is connected to a mobile terminal or a terminal device via a wireless communication function (not illustrated) conforming to a standard such as Bluetooth (registered trademark) or WiFi (registered trademark). The communication function of the gait information generation device 10 may conform to a standard other than Bluetooth (registered trademark) or WiFi (registered trademark). The gait information may be used by an application installed in a mobile terminal or a terminal device. In this case, the mobile terminal or the terminal device executes processing using the gait information by application software or the like installed in the device. The gait information generation device 10 may be mounted on a mobile terminal or a terminal device.

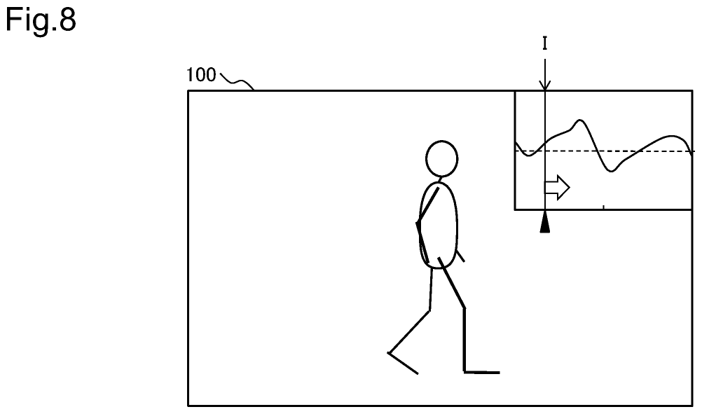

FIG. 8 is a conceptual diagram illustrating an example in which the gait information output from the gait information generation device 10 is superimposed on the video in which the gait subject is displayed and displayed. In the example of FIG. 8, a graph indicating the time-series data of the waist oscillation is superimposed on the video in which the gait subject is displayed and displayed. In the example of FIG. 8, the waist of the subject is displayed at the center of the screen 100. On the upper right of the screen 100, a graph showing time-series data of the waist oscillation is displayed. The display position of the graph indicating the time-series data of the waist oscillation may be a region other than the upper right of the screen 100. The graph showing the time-series data of the waist oscillation shows the indicator I (vertical line) displayed at a position corresponding to the gait phase of the subject.

As illustrated in FIG. 8, when the graph of the waist oscillation is displayed on the screen 100, it is easy to intuitively grasp the waist oscillation of the subject in accordance with the gait of the subject displayed at the center of the screen 100. For example, an actually captured video is used as the video. For example, a virtual person (character) that operates in accordance with gait of the subject may be used for the video. Not the entire body of the subject but only a portion below the waist (lower body) may be displayed on the video.

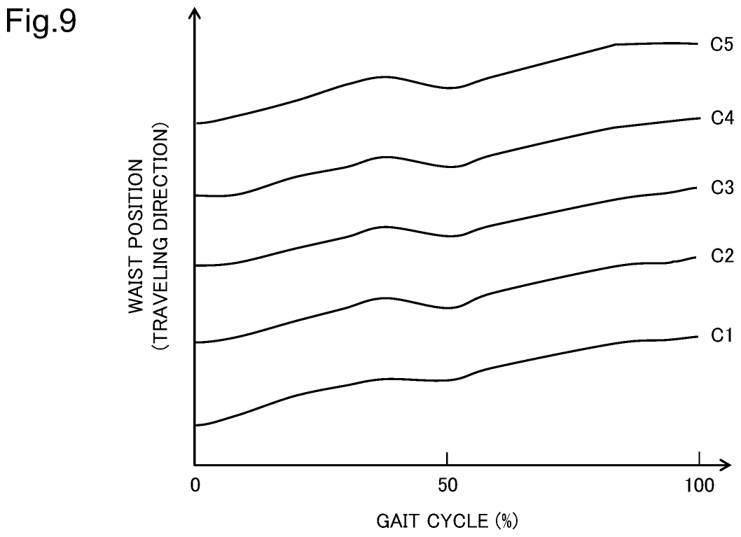

FIG. 9 is a graph for describing an example of time-series data of a waist position in a traveling direction regarding a plurality of gait cycles. FIG. 9 illustrates time-series data C1 to C5 of the waist position in the traveling direction in a plurality of consecutive gait cycles. The waist position lapses in the order of the time-series data C1, the time-series data C2, the time-series data C3, the time-series data C4, and the time-series data C5. With respect to the time-series data of the consecutive waist positions, the waist position of the ending point of the preceding time-series data and the waist position of the starting point of the subsequent time-series data match. The shapes of the time-series data C1 to C5 of the waist position change according to the gait cycle.

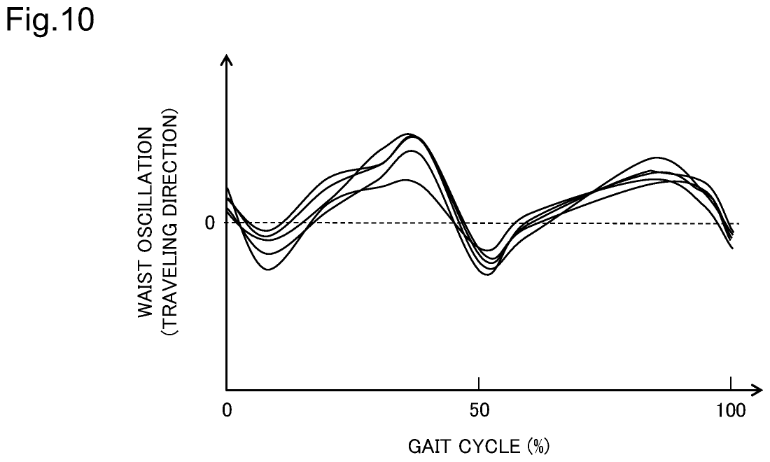

FIG. 10 is a graph illustrating an example of the gait information generated based on the time-series data of the waist position of FIG. 9. FIG. 10 is an example in which time-series data of the waist oscillation regarding a plurality of gait cycles is displayed side by side in association with the gait cycle. According to the graph of FIG. 10, it is possible to intuitively grasp the change in the waist oscillation regarding the plurality of gait cycles in association with the gait cycle. According to the graph of FIG. 10, the movement of the subject over a plurality of gait cycles can be verified. For example, statistical values such as an arithmetic mean, a geometric mean, a variance, and a standard deviation of the waist oscillation regarding a plurality of gait cycles may be derived. By using an average value such as an arithmetic mean or a geometric mean of the waist oscillation regarding the plurality of gait cycles, the movement of the waist in the plurality of gait cycles can be grasped on average. By using the dispersion, the standard deviation, or the like of the waist oscillation regarding the plurality of gait cycles, it is possible to grasp the fluctuation of the movement of the waist in the plurality of gait cycles.

(Operation)

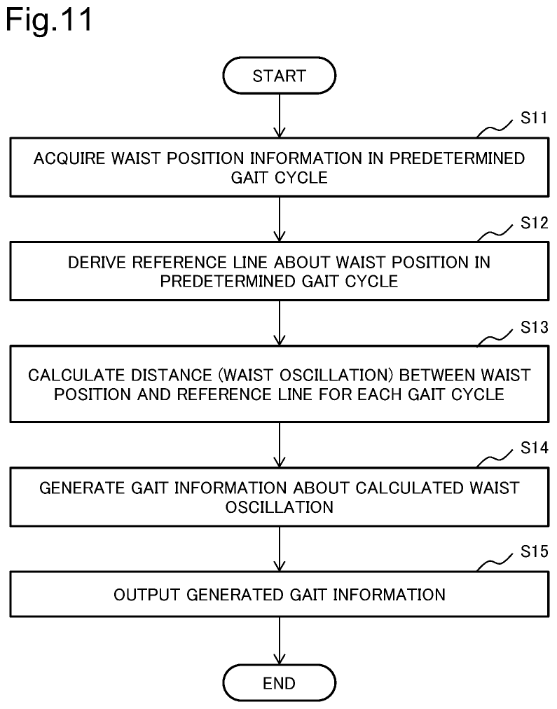

Next, an example of an operation of the gait information generation device 10 will be described with reference to the drawings. FIG. 11 is a flowchart for describing an example of an operation of the gait information generation device 10. In the description along the flowchart of FIG. 11, the gait information generation device 10 is an operation subject.

In FIG. 11, first, the gait information generation device 10 acquires waist position information in a predetermined gait cycle (step S11). For example, the gait information generation device 10 acquires waist position information in one gait cycle. The gait information generation device 10 may acquire waist position information in a plurality of gait cycles.

Next, the gait information generation device 10 derives a reference line regarding the waist position in the predetermined gait cycle (step S12). For example, the gait information generation device 10 derives a reference line regarding the waist position of one gait cycle. The gait information generation device 10 may derive a reference line regarding the waist position in a plurality of gait cycles.

Next, the gait information generation device 10 calculates a distance (waist oscillation) between the waist position and the reference line for each gait cycle (step S13).

Next, the gait information generation device 10 generates the gait information about the calculated waist oscillation (step S14). For example, the gait information generation device 10 generates the gait information including the time-series data of the waist oscillation and the graph of the time-series data of the waist oscillation.

Next, the gait information generation device 10 outputs the generated gait information (step S15). For example, the gait information generation device 10 outputs the gait information including the time-series data of the waist oscillation and the graph of the time-series data of the waist oscillation.

### Application Example

Next, an application example regarding the gait information generation device 10 will be described with reference to the drawings. Here, an example in which the gait information output from the gait information generation device 10 is displayed on a screen of a terminal device will be described. In the following application example, an example in which gait information is displayed on a video of a gait subject will be described. The video of the subject may be an actual video or a virtual person (character) imitating the motion of the subject. Hereinafter, an example in which a character is displayed in a video will be described. The display information illustrated in the following application example may be generated by the gait information generation device 10, or may be generated by another device or system that has acquired the gait information.

### Application Example 1-1

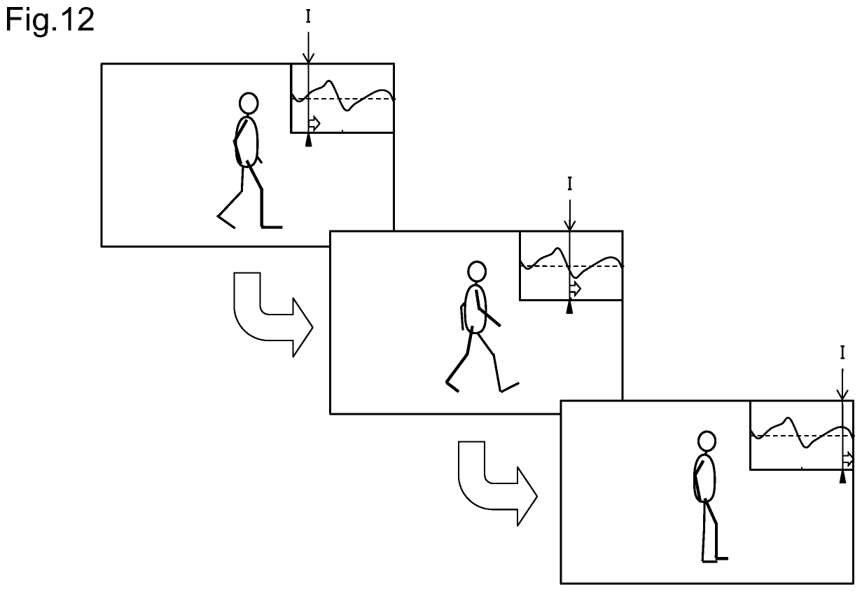

FIG. 12 is a conceptual diagram related to Application Example 1-1 related to the gait information generation device 10. In Application Example 1-1, as the gait information output from the gait information generation device 10, a graph of the time-series data of the waist oscillation is displayed in a frame constituting a video regarding gait of the subject. In the example of FIG. 12, the graph of the time-series data of the waist oscillation is displayed in the upper right region of the screen. The graph of the time-series data of the waist oscillation may be displayed in a region other than the upper right region of the screen.

FIG. 12 illustrates three frames extracted from a plurality of frames included in a video related to gait of the subject. The actual video is composed of more frames. In the three frames in FIG. 12, time (gait cycle) progresses from the upper left to the lower right. In the graph of the time-series data of the waist oscillation displayed in each frame, an indicator I (vertical line) is displayed at a position corresponding to the gait phase of the subject. The indicator I moves in the direction indicated by the arrow in accordance with the gait phase of the subject.

In the present application example, a graph of the time-series data of the waist oscillation is displayed in association with the gait phase of the subject. According to the present application example, the gait can be intuitively grasped based on the graph of the time-series data of the waist oscillation associated with the gait of the subject. Specifically, according to the present application example, the gait can be intuitively grasped according to the movement of the indicator I displayed in the graph of the time-series data of the waist oscillation.

### Application Example 1-2

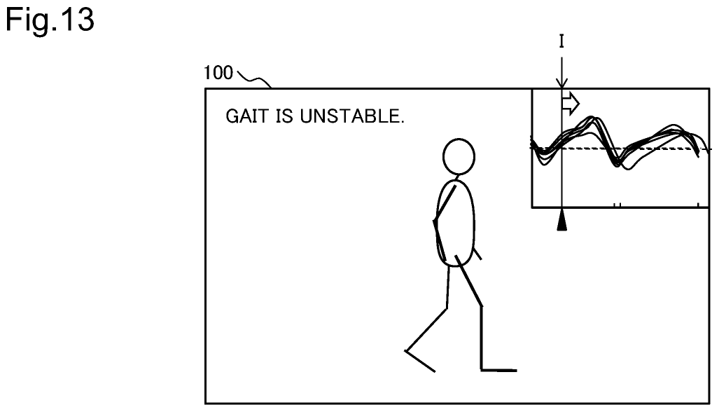

FIG. 13 is a conceptual diagram related to Application Example 1-2 related to the gait information generation device 10. In Application Example 1-2, as the gait information output from the gait information generation device 10, a graph of the time-series data of the waist oscillation in a plurality of gait cycles is displayed in a frame constituting a video regarding gait of the subject. In the example of FIG. 13, a graph of the time-series data of the waist oscillation in a plurality of gait cycles is displayed in the upper right region of the screen. The graph of the time-series data of the waist oscillation in the plurality of gait cycles may be displayed in a region other than the upper right region of the screen.

FIG. 13 illustrates one frame extracted from a plurality of frames included in a video related to gait of the subject. The actual video is composed of a plurality of frames. In the graph of the time-series data of the waist oscillation displayed in the frame, an indicator I (vertical line) is displayed at a position corresponding to the gait phase of the subject. The indicator I moves in the direction indicated by the arrow in accordance with the gait phase of the subject.

In FIG. 13, information according to the fluctuation of time-series data of the waist oscillation in a plurality of gait cycles is displayed. FIG. 13 illustrates an example in which the dispersion or the standard deviation of the waist oscillation in a plurality of gait cycles is increased in accordance with gait. On the screen 100, information that “gait has become unstable” is displayed according to the increase in the dispersion or the standard deviation of the waist oscillation. For example, in a case where the gait of the subject is displayed on the screen 100 in real time, recommendation information for the subject may be displayed according to an increase in the dispersion or the standard deviation of the waist oscillation. For example, recommendation information that recommends a break or recommends correction of gait may be displayed according to an increase in the dispersion or the standard deviation of the waist oscillation.

In the present application example, a graph of the time-series data of the waist oscillation in a plurality of gait cycles is displayed in association with the gait phase of the subject. According to the present application example, it is possible to intuitively grasp the gait over the plurality of gait cycles based on the graph of the time-series data of the waist oscillation in the plurality of gait cycles associated with the gait of the subject. According to the present application example, by displaying the information according to the fluctuation of the dispersion or the standard deviation of the waist oscillation, it is possible to intuitively grasp the change in the gait according to the continuation of gait.

### Application Example 1-3

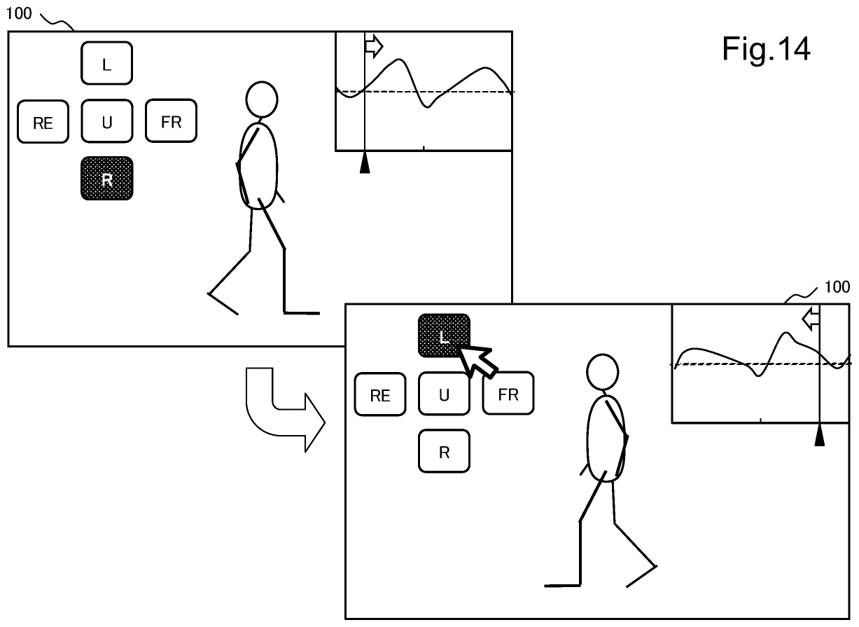

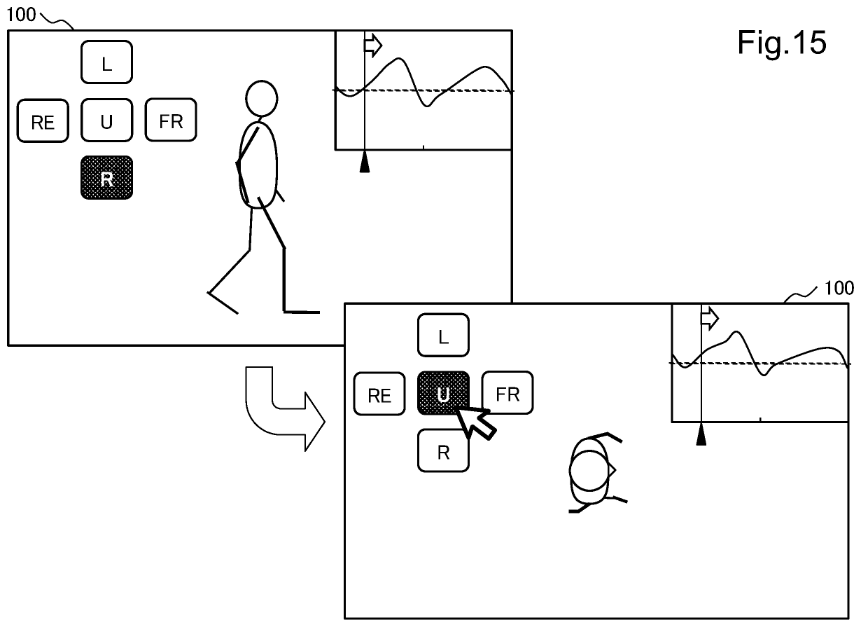

FIGS. 14 to 15 are conceptual diagrams related to Application Example 1-3 related to the gait information generation device 10. In Application Example 1-3, the viewpoint of the virtual camera set for the character in the frame is switched according to the operation of the subject. In Application Example 1-3, as the gait information output from the gait information generation device 10, a graph of the time-series data of the waist oscillation is displayed in a frame constituting a video regarding gait of the subject. In FIGS. 14 to 15, graphs of time-series data of the waist oscillation are displayed in an upper right region of the screen. The graph of the time-series data of the waist oscillation in the plurality of gait cycles may be displayed in a region other than the upper right region of the screen. In FIGS. 14 to 15, the direction of the graph is changed in accordance with the viewpoint of the virtual camera.

In the examples of FIGS. 14 to 15, a button for changing the viewpoint of the virtual camera is displayed at the upper left of the screen. In the examples of FIGS. 14 to 15, the viewpoint of the virtual camera can be set to the rear RE, the left L, the right R, the upper U, and the front FR. The position where the viewpoint of the virtual camera is set and the arrangement of the buttons are not limited to the examples of FIGS. 14 to 15.

FIG. 14 illustrates an example in which the button for changing the viewpoint of the virtual camera to the left L is selected in a state (upper left) in which the viewpoint of the virtual camera is set to the right R. In response to selection of the button for changing the viewpoint of the virtual camera to the left L, the viewpoint of the virtual camera changes to a state set to the left L (lower right). In the example of FIG. 14, the direction of the horizontal axis of the graph is changed to the opposite direction in accordance with the traveling direction of the subject. In the example of FIG. 14, the direction in which the indicator I (vertical line) associated with the gait phase moves is also changed to the opposite direction in accordance with the traveling direction of the subject. The display position of the button or the graph may be changed in response to selection of the button for changing the viewpoint of the virtual camera.

FIG. 15 illustrates an example in which the button for changing the viewpoint of the virtual camera to the upper U is selected in a state (upper left) in which the viewpoint of the virtual camera is set to the right R. In response to selection of the button for changing the viewpoint of the virtual camera to the upper U, the viewpoint of the virtual camera is changed to a state (lower right) in which the viewpoint of the virtual camera is set to the upper U. In the example of FIG. 15, since the traveling direction of the subject on the screen 100 does not change, the direction of the horizontal axis of the graph is not changed. In the example of FIG. 15, the direction in which the indicator I (vertical line) associated with the gait phase moves is not changed. The display position of the button or the graph may be changed in response to selection of the button for changing the viewpoint of the virtual camera.

In the present application example, the viewpoint of the virtual camera is changed according to the operation of the subject. According to the present application example, since gait can be verified from a plurality of viewpoints by changing the viewpoint of the virtual camera, the gait can be more intuitively grasped.

As described above, the gait information generation device of the present example embodiment includes the acquisition unit, the waist oscillation calculation unit, the gait information generation unit, and the output unit. The acquisition unit acquires waist position information including time-series data of a waist position of the subject in a predetermined gait cycle. The waist oscillation calculation unit calculates the waist oscillation for a plurality of gait phases included in the predetermined gait cycle. The waist oscillation corresponds to the distance between the reference line set for the time-series data of the waist position of the subject and the waist position in each of the plurality of gait phases. The gait information generation unit generates the gait information according to the waist oscillation calculated with respect to the predetermined gait cycle. The output unit outputs the generated gait information.

By displaying the gait information (display information) in a moving image in conjunction with the gait of the subject, the gait can be grasped more intuitively. For example, when the viewpoint (following center) of the virtual camera is caused to follow the waist of the subject, the gait can be intuitively captured by the movement of the foot of the subject. However, in a case where the following center is caused to follow the waist of the subject, although the movement of the foot can be intuitively grasped, acceleration/deceleration in gait cannot be intuitively grasped.

In the present example embodiment, the gait information according to the waist oscillation of the subject is generated. Therefore, according to the present example embodiment, the gait of the subject can be intuitively grasped by the gait information including the information about the waist oscillation that fluctuates according to the gait of the subject.

In an aspect of the present example embodiment, the acquisition unit acquires waist position information including time-series data of a waist position of the subject in one gait cycle. The waist oscillation calculation unit calculates waist oscillation corresponding to a distance between a reference line set for the time-series data of the waist position of the subject and the waist position in each of the plurality of gait phases for a plurality of gait phases included in one gait cycle. The gait information generation unit generates the gait information including information according to the fluctuation of the waist oscillation calculated with respect to one gait cycle. According to the present aspect, by the gait information according to the waist oscillation in one gait cycle, the gait of the subject in the gait cycle can be intuitively grasped.

In an aspect of the present example embodiment, the acquisition unit acquires waist position information including time-series data of a waist position of the subject in a plurality of gait cycles. The waist oscillation calculation unit calculates the waist oscillation corresponding to a distance between the reference line set for the time-series data of the waist position of the subject in each of the plurality of gait cycles and the waist position in each of the plurality of gait phases for a plurality of gait phases included in the plurality of gait cycles. The gait information generation unit generates the gait information including information according to the fluctuation of the waist oscillation calculated for each of the plurality of gait cycles. According to the present aspect, by the gait information according to the waist oscillation in a plurality of gait cycles, it is possible to intuitively grasp the fluctuation in the gait of the subject in the plurality of gait cycles.

In an aspect of the present example embodiment, the gait information generation unit generates, as the gait information, display information in which time-series data of the waist oscillation in a predetermined gait cycle is superimposed on a frame constituting a video indicating a gait state of the subject. According to the present aspect, the gait of the subject can be more intuitively grasped in accordance with the video in which the subject walks by the time-series data of the waist oscillation displayed in the frame constituting the video.

In an aspect of the present example embodiment, the gait information generation unit generates the display information in which the indicator associated with the gait phase of the subject is superimposed on the time-series data of the waist oscillation in the predetermined gait cycle. According to the present aspect, it is possible to more intuitively grasp the gait of the subject in accordance with the video in which the subject walks by the indicator superimposed on the time-series data of the waist oscillation.

In the present example embodiment, an example in which the gait information about the waist oscillation according to the motion of the person (subject) in the real world is generated is described. The method of the present example embodiment may be applied to generation of the gait information about the waist oscillation according to the motion of the virtual person such as the avatar in the virtual world. When the avatar is operated by the movement according to the waist oscillation, the movement of the avatar can be more realistically expressed. The method of the present example embodiment may be applied to generation of the gait information about the oscillation of the site other than the waist. The method of the present example embodiment may be used for verification of waist oscillation other than gait.

### Second Example Embodiment

Next, a gait information generation device according to the second example embodiment will be described with reference to the drawings. The present example embodiment is different from the first example embodiment in that speed information according to the waist oscillation is calculated. Hereinafter, the description of the similar configuration and function as those of the first example embodiment may be omitted.

(Configuration)

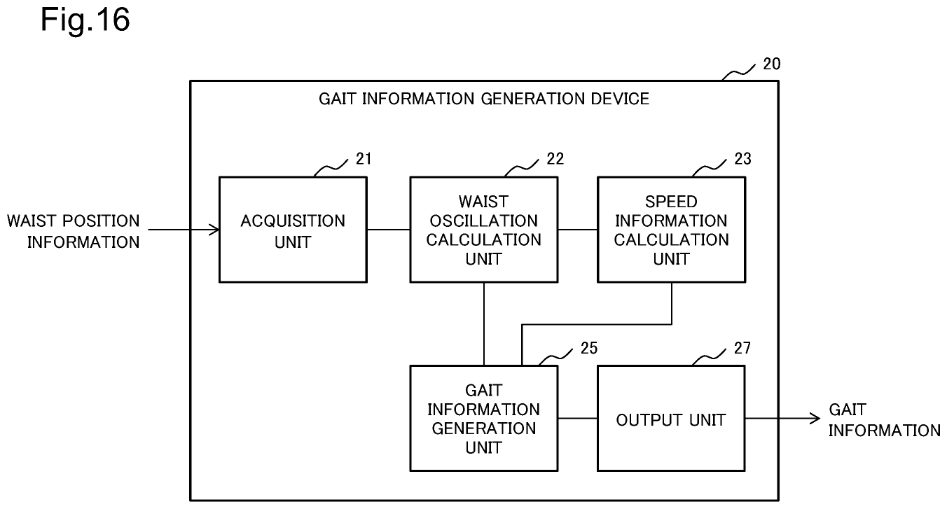

FIG. 16 is a block diagram illustrating a configuration of a gait information generation device 20 according to the present example embodiment. The gait information generation device 20 includes an acquisition unit 21, a waist oscillation calculation unit 22, a speed information calculation unit 23, a gait information generation unit 25, and an output unit 27.

The acquisition unit 21 has the similar configuration as the acquisition unit 11 of the first example embodiment. The acquisition unit 21 acquires waist position information about the subject in the traveling direction. The acquisition unit 21 acquires waist position information in the predetermined gait section.

The waist oscillation calculation unit 22 has the similar configuration as the waist oscillation calculation unit 12 of the first example embodiment. The waist oscillation calculation unit 22 acquires the waist position information about the subject from the acquisition unit 21. The waist oscillation calculation unit 22 calculates a position difference (waist oscillation) between the position of the subject and the waist position according to the average speed using the acquired waist position information. The waist oscillation calculation unit 22 calculates the waist oscillation based on the position of the subject according to the average speed in the predetermined gait section.

The speed information calculation unit 23 acquires the waist oscillation calculated by the waist oscillation calculation unit 22. The speed information calculation unit 23 calculates speed information according to the acquired waist oscillation. For example, the speed information calculation unit 23 calculates speed information according to the inclination (speed) of the tangent in each gait phase of the curve indicating the time-series data of the waist oscillation. For example, the speed information calculation unit 23 calculates the speed information according to the rate of change (speed) in the waist oscillation in the minute section of the curve indicating the time-series data of the waist oscillation.

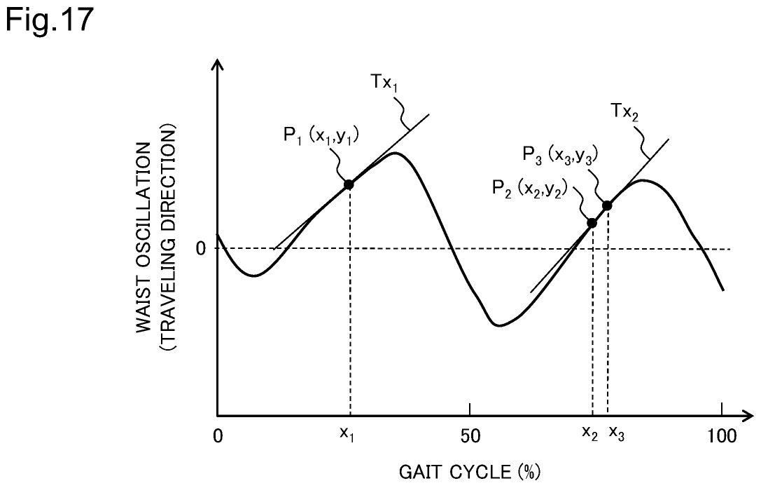

FIG. 17 is a graph for explaining the speed information calculated by the speed information calculation unit 23. For example, the speed information calculation unit 23 calculates the inclination of a tangent Tx1 at the point P1 (x1, y1) of a gait phase x1 as the speed of the waist oscillation with respect to the curve indicating the time-series data of the waist oscillation. For example, the speed information calculation unit 23 calculates the rate of change in the waist oscillation in the minute section between a point P2 (x2, y2) of a gait phase x2 and a point P3 (x3, y3) of a gait phase x3 as the speed of the waist oscillation with respect to the curve indicating the time-series data of the waist oscillation. The rate of change in the waist oscillation in the minute section between the point P2 (x2, y2) and the point P23 (x3, y3) corresponds to the inclination of a straight line Tx2 passing through the point P2 (x2, y2) and the point P23 (x3, y3).

The gait information generation unit 25 acquires speed information about the waist oscillation calculated by the speed information calculation unit 23. The gait information generation unit 25 generates the gait information according to the speed information about the waist oscillation. For example, the gait information generation unit 25 generates, as the gait information, time-series data in which the direction and the magnitude of the speed regarding the waist oscillation are associated with the gait phase. The gait information may include information about the waist oscillation calculated by the waist oscillation calculation unit 22. In this case, the gait information generation unit 25 acquires the waist oscillation calculated by the waist oscillation calculation unit 22. For example, the gait information generation unit 25 generates the gait information including the time-series data of the waist oscillation and the graph indicating the time-series data of the waist oscillation. The gait information generated by the gait information generation unit 25 is not limited to the direction and the magnitude of the speed related to the waist oscillation, and the time-series data and the graph of the waist oscillation.

The output unit 27 has the similar configuration as the output unit 17 of the first example embodiment. The output unit 27 outputs the gait information generated by the gait information generation unit 25. The use of the gait information output from the output unit 27 is not particularly limited.

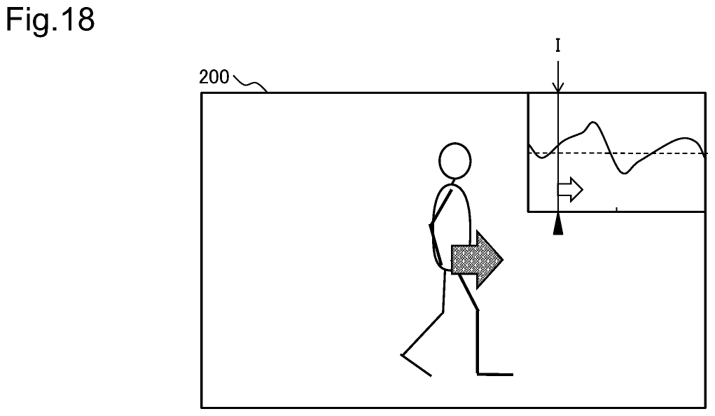

FIG. 18 is a conceptual diagram illustrating an example in which the gait information output from the gait information generation device 20 is superimposed on the video in which the gait subject is displayed and displayed. FIG. 18 illustrates an example in which an arrow (also referred to as an index) indicating the direction and the magnitude of the speed of the waist oscillation is displayed in association with the subject of the screen 200. In the example of FIG. 18, the waist of the subject is displayed at the center of a screen 200. As in the first example embodiment, a graph showing time-series data of the waist oscillation is displayed on the upper right of the screen 200. The display position of the graph indicating the time-series data of the waist oscillation may be a region other than the upper right region of the screen 200. The graph showing the time-series data of the waist oscillation shows the indicator I (vertical line) displayed at a position corresponding to the gait phase of the subject. The graph showing the time-series data of the waist oscillation may be omitted. A graph showing time-series data of the speed of the waist oscillation may be displayed on the screen 200.

As illustrated in FIG. 18, by displaying the arrow indicating the direction and the magnitude of the speed related to the waist oscillation on the screen 200, it is easy to intuitively grasp the speed of the waist oscillation of the subject in accordance with the gait of the subject displayed at the center of the screen 200. The direction and the magnitude of the speed of the waist oscillation may be expressed by the thickness, color, shade, pattern, and the like of the arrow. When the direction and the magnitude of the speed of the waist oscillation are expressed by the thickness, color, shade, pattern, and the like of the arrow, the fluctuation of the waist oscillation can be more visually grasped.

(Operation)

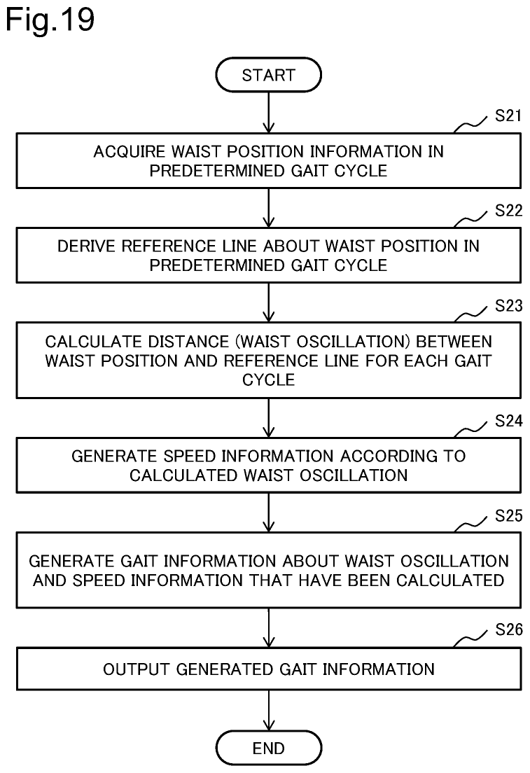

Next, an example of an operation of the gait information generation device 20 will be described with reference to the drawings. FIG. 19 is a flowchart for describing an example of an operation of the gait information generation device 20. In the description along the flowchart of FIG. 19, the gait information generation device 20 is an operation subject.

In FIG. 19, first, the gait information generation device 20 acquires waist position information in a predetermined gait cycle (step S21). For example, the gait information generation device 20 acquires waist position information in one gait cycle. The gait information generation device 20 may acquire waist position information in a plurality of gait cycles.

Next, the gait information generation device 20 derives a reference line regarding the waist position in the predetermined gait cycle (step S22). For example, the gait information generation device 20 derives a reference line regarding the waist position of one gait cycle. The gait information generation device 20 may derive a reference line regarding the waist position in a plurality of gait cycles.

Next, the gait information generation device 20 calculates a distance (waist oscillation) between the waist position and the reference line for each gait cycle (step S23).

Next, the gait information generation device 20 generates speed information according to the calculated waist oscillation (step S24). For example, the gait information generation device 20 generates speed information indicating the magnitude and the direction of the speed regarding the waist oscillation.

Next, the gait information generation device 20 generates the gait information about the calculated waist oscillation and speed information (step S25). For example, the gait information generation device 20 generates the gait information including an arrow (index) indicating the magnitude and the direction of the speed related to the waist oscillation. For example, the gait information generation device 20 generates the gait information including the time-series data of the waist oscillation and the graph of the time-series data of the waist oscillation.

Next, the gait information generation device 20 outputs the generated gait information (step S26). For example, the gait information generation device 20 outputs the gait information including an arrow (index) indicating the magnitude and the direction of the speed of the waist oscillation and a graph of the time-series data of the waist oscillation.

### Application Example

Next, an application example regarding the gait information generation device 20 will be described with reference to the drawings. Here, an example in which the gait information output from the gait information generation device 20 is displayed on a screen of a terminal device will be described. In the following application example, an example in which gait information is displayed on a video of a gait subject will be described. The image of the subject may be an actual video or a virtual person (character). Hereinafter, an example in which a character is displayed in a video will be described. The display information illustrated in the following application example may be generated by the gait information generation device 20, or may be generated by another device or system that has acquired the gait information.

### Application Example 2-1

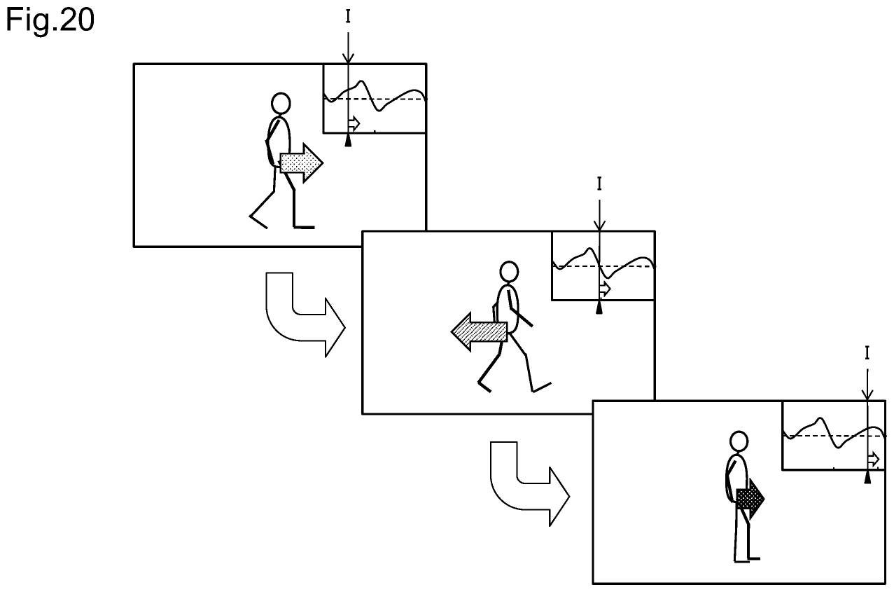

FIG. 20 is a conceptual diagram related to Application Example 2-1 related to the gait information generation device 20. In Application Example 2-1, as the gait information output from the gait information generation device 20, an arrow corresponding to the speed information about the waist oscillation is displayed on a frame constituting a video regarding gait of the subject. The arrow corresponding to the speed information about the waist oscillation is displayed along the waist position of the character in the frame. The arrow corresponding to the speed information about the waist oscillation may be displayed at a position different from the waist position. For example, the arrow corresponding to the speed information about the waist oscillation may be displayed ahead of or behind the character in the frame according to the direction of the speed of the waist oscillation. In the example of FIG. 20, as in the first example embodiment, a graph of the time-series data of the waist oscillation is displayed in the upper right region of the frame. The graph of the time-series data of the waist oscillation may be omitted. In the frame, a graph of the time-series data regarding the speed of the waist oscillation may be displayed.

FIG. 20 illustrates three frames extracted from a plurality of frames included in a video related to gait of the subject. The actual video is composed of more frames. In the three frames in FIG. 20, time (gait cycle) progresses from the upper left to the lower right. An arrow corresponding to speed information about the waist oscillation is displayed at the waist position of the character in the frame. The arrow has a shape that reflects a direction and a magnitude of the speed of the waist oscillation. The direction of the arrow is directed to the direction of the speed of the waist oscillation. The magnitude of the arrow is set to a length corresponding to the speed of the waist oscillation. The higher the speed of the waist oscillation is, the longer the length of the arrow is, and the lower the speed of the waist oscillation is, the shorter the length of the arrow. In the graph of the time-series data of the waist oscillation displayed in the frame, an indicator I (vertical line) is displayed at a position corresponding to the gait phase of the subject. The indicator I moves in the direction indicated by the arrow in accordance with the gait phase of the subject. The arrow displayed at the waist position of the character in the frame indicates speed information about the waist oscillation at the indicator I of the graph.

In the upper left frame, since the speed of the waist oscillation is positive, the direction of the arrow corresponding to the speed information about the waist oscillation is forward. In the center frame, since the speed of the waist oscillation is negative, the direction of the arrow corresponding to the speed information about the waist oscillation is backward. In the upper left frame and the center frame, the directions of the waist oscillation are opposite, so that the directions of the arrow are opposite. The absolute value of the speed of the waist oscillation in the central frame is larger than that in the upper left frame. Therefore, the length of the arrow in the central frame is longer than that in the upper left frame. In the lower right frame, since the speed of the waist oscillation is positive, the direction of the arrow corresponding to the speed information about the waist oscillation is forward. The speed of the waist oscillation in the lower right frame is lower that in the upper left frame. Therefore, the length of the arrow in the lower right frame is shorter than that in the upper left frame. When the speed of the waist oscillation is zero, the arrow corresponding to the speed information about the waist oscillation is not displayed. When the speed of the waist oscillation is zero, a symbol or a figure other than an arrow may be displayed.

In the present application example, an arrow corresponding to the speed information about the waist oscillation is displayed in association with the gait phase of the subject. According to the present application example, the gait can be intuitively grasped based on the arrow corresponding to the speed information about the waist oscillation associated with the gait of the subject. Specifically, according to the present application example, the gait can be intuitively grasped according to the change in the direction and the length of the arrow corresponding to the speed information about the waist oscillation.

As described above, the gait information generation device of the present example embodiment includes the acquisition unit, the waist oscillation calculation unit, the speed information generation unit, the gait information generation unit, and the output unit. The acquisition unit acquires waist position information including time-series data of a waist position of the subject in a predetermined gait cycle. The waist oscillation calculation unit calculates the waist oscillation corresponding to a distance between a reference line set for the time-series data of the waist position of the subject and the waist position in each of the plurality of gait phases for a plurality of gait phases included in the predetermined gait cycle. The speed information generation unit generates speed information corresponding to a direction and a magnitude of the speed of the waist oscillation. The gait information generation unit generates the gait information according to the waist oscillation calculated with respect to the predetermined gait cycle. The gait information generation unit generates, as the gait information, display information in which an arrow indicating a direction and a magnitude of the speed of the waist oscillation is superimposed on a frame constituting a video indicating the gait state of the subject. The output unit outputs the gait information including the generated display information.

In the present example embodiment, the gait information including the arrow corresponding to a direction and a magnitude of the speed of the waist oscillation is generated for the subject. Therefore, according to the present example embodiment, the gait of the subject can be more intuitively grasped by the arrow corresponding to the speed of the waist oscillation due to gait of the subject.

### Third Example Embodiment

Next, a gait information generation device according to the third example embodiment will be described with reference to the drawings. The present example embodiment is different from the first to second example embodiments in that a viewpoint (following center) of a virtual camera is changed according to the waist oscillation. Hereinafter, an example in which a configuration in which the following center of the virtual camera is changed is added to the configuration of the first example embodiment will be described. The configuration in which the following center of the virtual camera is changed may be added to the configuration of the second example embodiment. Hereinafter, the description of the similar configuration and function as those of the first to second example embodiments may be omitted.

(Configuration)

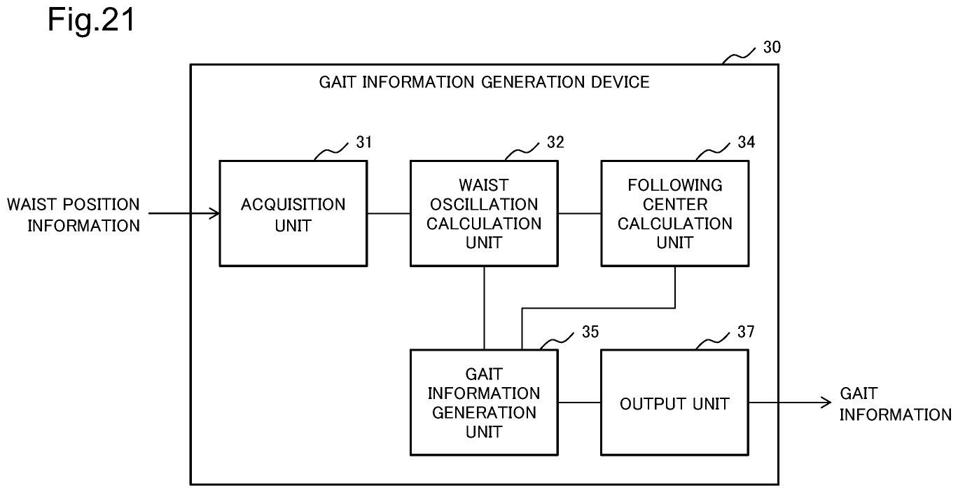

FIG. 21 is a block diagram illustrating a configuration of a gait information generation device 30 according to the present example embodiment. The gait information generation device 30 includes an acquisition unit 31, a waist oscillation calculation unit 32, a following center calculation unit 34, a gait information generation unit 35, and an output unit 37.

The acquisition unit 31 has the similar configuration as the acquisition unit 11 of the first example embodiment. Acquisition unit 31 acquires waist position information about the subject in the traveling direction. Acquisition unit 31 acquires waist position information in the predetermined gait section.

The waist oscillation calculation unit 32 has the similar configuration as the waist oscillation calculation unit 12 of the first example embodiment. The waist oscillation calculation unit 32 acquires the waist position information about the subject from the acquisition unit 31. The waist oscillation calculation unit 32 calculates a position difference (waist oscillation) between the position of the subject according to the average speed and the waist position using the acquired waist position information. The waist oscillation calculation unit 32 calculates the waist oscillation based on the position of the subject according to the average speed in the predetermined gait section.

The following center calculation unit 34 acquires the waist oscillation calculated by the waist oscillation calculation unit 32. The following center calculation unit 34 calculates the position of the viewpoint (following center) of the virtual camera according to the acquired waist oscillation. The following center corresponds to the center of a plurality of frames constituting a video. The following center is set at the center of the screen on which the video related to the gait of the subject is displayed. A difference between the waist position and the following center corresponds to waist oscillation.

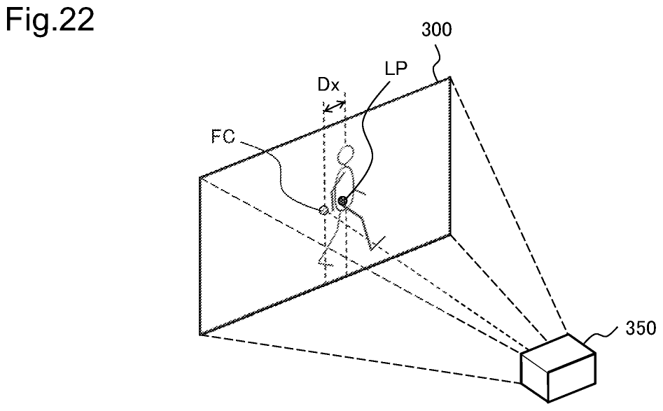

FIG. 22 is a conceptual diagram illustrating an example in which a gait subject is displayed on a screen 300. A viewpoint (following center FC) of a virtual camera 350 that follows the subject is set at a position that captures the center of the screen 300. On the screen 300, the subject is displayed at a position corresponding to the waist oscillation Dx that is a difference between the waist position LP and the following center FC. In the example of FIG. 22, the positions of the following center FC and the waist position LP are different.

The gait information generation unit 35 generates the gait information corresponding to a difference between the position of the following center calculated by the following center calculation unit 34 and the waist oscillation calculated by the waist oscillation calculation unit 32. For example, the gait information generation unit 35 generates the display information in which the waist position is set at a position shifted from the following center by the amount of the waist oscillation. The gait information generation unit 35 may generate the gait information including time-series data of the waist oscillation and a graph indicating the time-series data of the waist oscillation. The gait information generation unit 35 may generate the gait information including time-series data in which the direction and the magnitude of the speed related to the waist oscillation are associated with the gait phase. The gait information generated by the gait information generation unit 35 is not limited to the display information reflecting a difference (waist oscillation) between the waist position and the following center, the direction and the magnitude of the speed related to the waist oscillation, and the time-series data and the graph of the waist oscillation.

The output unit 37 has the similar configuration as the output unit 17 of the first example embodiment. The output unit 37 outputs the gait information generated by the gait information generation unit 35. The use of the gait information output from the output unit 37 is not particularly limited.

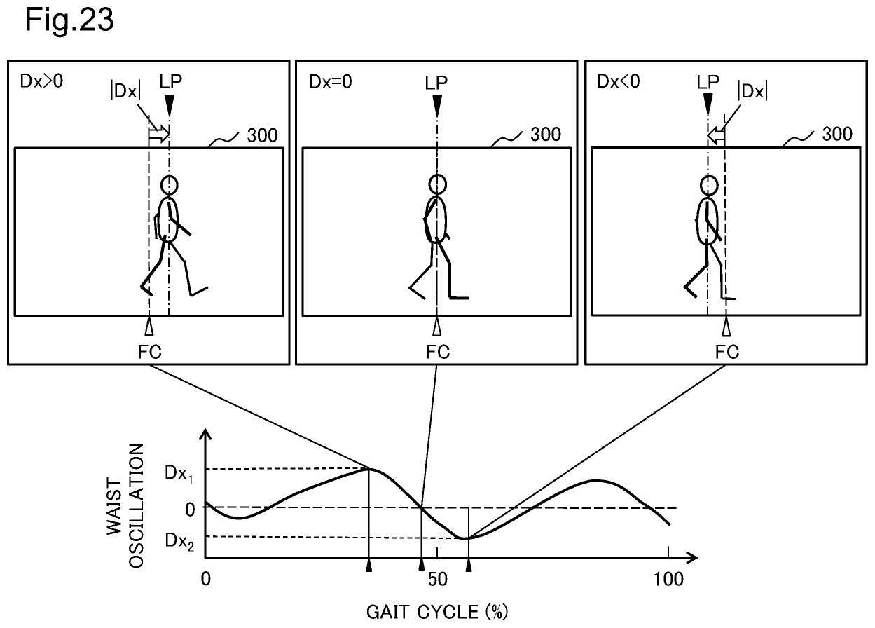

FIG. 23 is a conceptual diagram for describing a display position of the subject according to the waist oscillation. When waist oscillation Dx is positive (left), the following center FC is delayed with respect to the waist position LP. Therefore, when the waist oscillation Dx is positive (left), the subject is displayed at a position shifted rightward by the absolute value |Dx| of the waist oscillation Dx from the center (following center FC) of the screen 300. When the waist oscillation Dx is zero (center), the waist position LP matches the following center FC. Therefore, when the waist oscillation Dx is zero (center), the subject is displayed at the center position of the screen 300 where the waist position LP and the following center FC match. When the waist oscillation Dx is negative (right), the following center FC is ahead of the waist position LP. Therefore, when the waist oscillation Dx is negative (right), the subject is displayed at a position shifted leftward by the absolute value |Dx| of the waist oscillation Dx from the center (following center FC) of the screen 300.

As illustrated in FIG. 23, when the subject is displayed at a position corresponding to a difference (waist oscillation) between the waist position and the following center, it is easy to intuitively grasp acceleration/deceleration in gait of the subject.

(Operation)

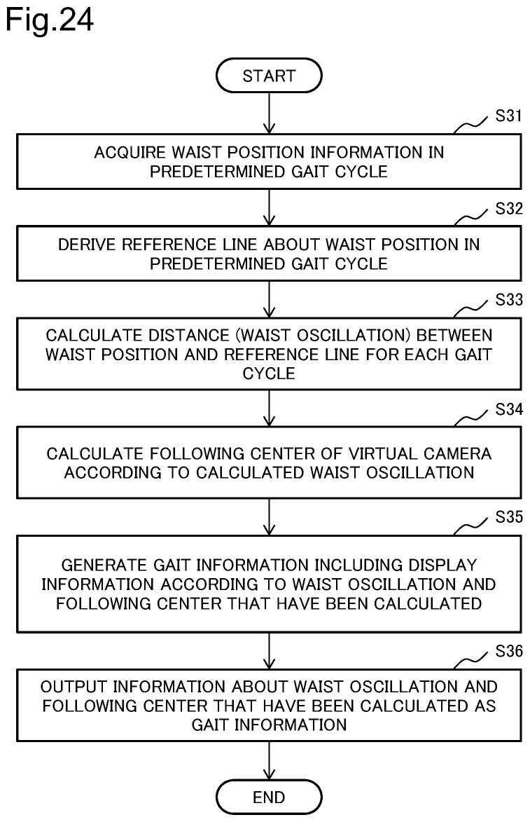

Next, an example of an operation of the gait information generation device 30 will be described with reference to the drawings. FIG. 24 is a flowchart for describing an example of an operation of the gait information generation device 30. In the description along the flowchart of FIG. 24, the gait information generation device 30 is an operation subject.

In FIG. 24, first, the gait information generation device 30 acquires waist position information in a predetermined gait cycle (step S31). For example, the gait information generation device 30 acquires waist position information in one gait cycle. The gait information generation device 30 may acquire waist position information in a plurality of gait cycles.

Next, the gait information generation device 30 derives a reference line regarding the waist position in the predetermined gait cycle (step S32). For example, the gait information generation device 30 derives a reference line regarding the waist position of one gait cycle. The gait information generation device 30 may derive a reference line regarding the waist position in a plurality of gait cycles.

Next, the gait information generation device 30 calculates a distance (waist oscillation) between the waist position and the reference line for each gait cycle (step S33).

Next, the gait information generation device 30 calculates a following center of the virtual camera 350 according to the calculated waist oscillation (step S34). For example, the gait information generation device 30 calculates the following center based on the waist position and the waist oscillation.

Next, the gait information generation device 30 generates the gait information including the display information according to the waist oscillation and the following center that have been calculated (step S35). For example, the gait information generation device 30 generates the gait information including the display information reflecting a difference (waist oscillation) between the waist position and the following center. For example, the gait information generation device 30 generates the gait information including the time-series data of the waist oscillation and the graph of the time-series data of the waist oscillation.

Next, the gait information generation device 30 outputs the generated gait information (step S36). For example, the gait information generation device 30 outputs gait information including display information reflecting a difference (waist oscillation) between the waist position and the following center and a graph of the time-series data of the waist oscillation.

### Application Example

Next, an application example regarding the gait information generation device 30 will be described with reference to the drawings. Here, an example in which the gait information output from the gait information generation device 30 is displayed on a screen of a terminal device will be described. In the following application example, an example in which gait information is displayed on a video of a gait subject will be described. The image of the subject may be an actual video or a virtual person (character). Hereinafter, an example in which a character is displayed in a video will be described. The display information illustrated in the following application example may be generated by the gait information generation device 30, or may be generated by another device or system that has acquired the gait information.

### Application Example 3-1

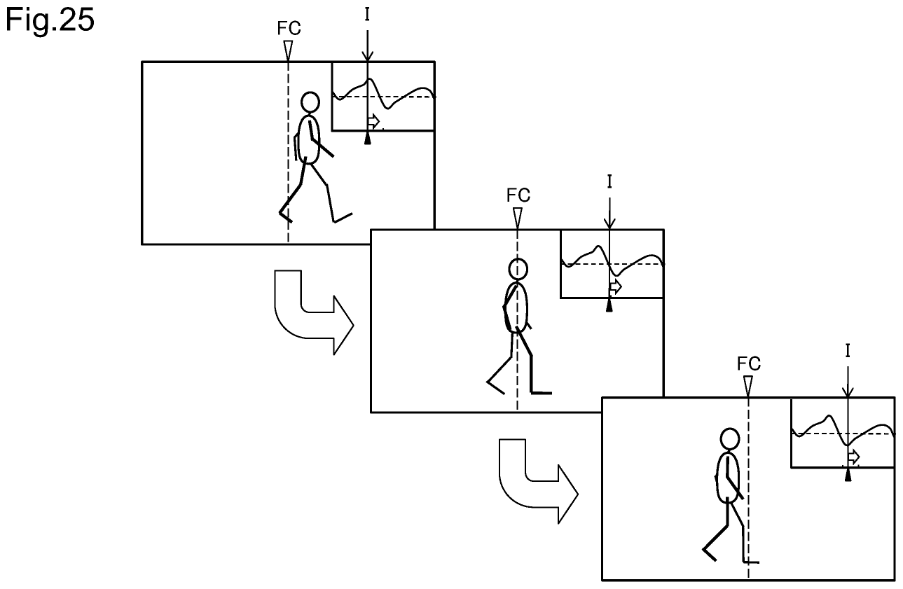

FIG. 25 is a conceptual diagram related to Application Example 3-1 related to the gait information generation device 30. In Application Example 3-1, display information in which the display position of the subject changes according to the waist oscillation of the subject is displayed as the gait information output from the gait information generation device 30. In the example of FIG. 25, as in the first example embodiment, a graph of the time-series data of the waist oscillation is displayed in the upper right region of the frame. The graph of the time-series data of the waist oscillation may be omitted. In the frame, a graph of the time-series data regarding the speed of the waist oscillation may be displayed.

FIG. 25 illustrates three frames extracted from a plurality of frames included in a video related to gait of the subject. The actual video is composed of more frames. In the three frames in FIG. 25, time (gait cycle) progresses from the upper left to the lower right. The character in the frame is displayed at a position corresponding to the waist oscillation. The character is displayed at a position corresponding to the direction of the waist oscillation with respect to the following center FC. It is displayed at a position corresponding to the magnitude of the waist oscillation. The larger the waist oscillation, the larger the shift amount of the character with respect to the following center FC. The smaller the waist oscillation, the smaller the shift amount of the character with respect to the following center FC. In the graph of the time-series data of the waist oscillation displayed in the frame, an indicator I (vertical line) is displayed at a position corresponding to the gait phase of the subject. The indicator I moves in the direction indicated by the arrow in accordance with the gait phase of the subject. The position of the character in the frame corresponds to the waist oscillation at the indicator I of the graph.

In the upper left frame, since the waist oscillation is positive, the character is displayed right of the following center FC. In the center frame, since the waist oscillation is zero, the character is displayed at the position of the following center FC. In the lower right frame, since the waist oscillation is negative, the character is displayed left side of the following center FC.

In the present application example, a character imitating the subject is displayed at a position corresponding to the direction and the magnitude of the waist oscillation in association with the gait phase of the subject. According to the present application example, the gait of the subject can be intuitively grasped based on the movement of the character displayed at the position corresponding to the direction and the magnitude of the waist oscillation.

### Application Example 3-2

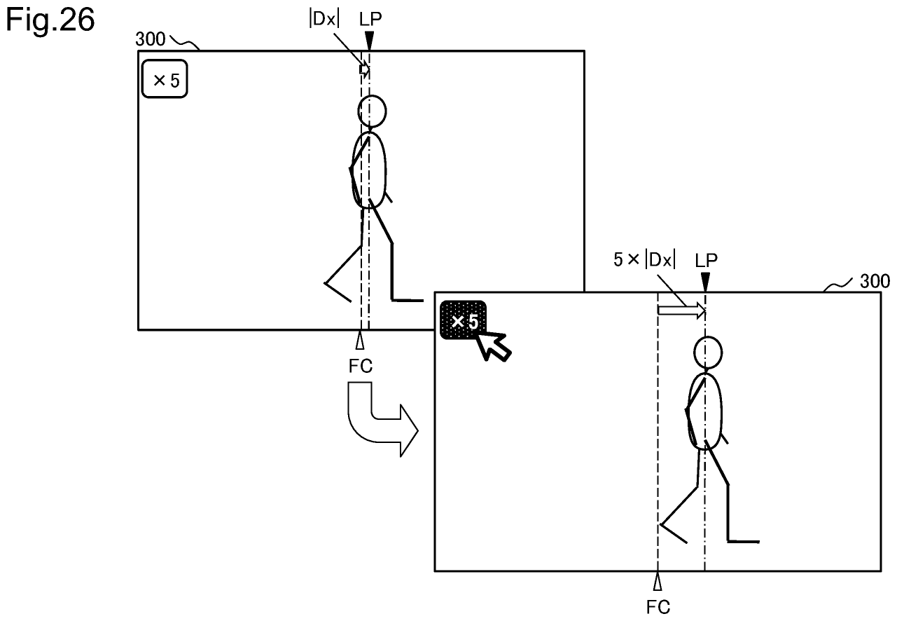

FIG. 26 is a conceptual diagram related to Application Example 3-2 related to the gait information generation device 30. In Application Example 3-2, the display position of the character according to the waist oscillation is switched according to the user's operation. In FIG. 26, in order to make the movement of the character clearer, the movement of the character is expressed with an amount of displacement larger than that of the actual waist oscillation.

In the example of FIG. 26, a button for changing the amount of displacement of the subject on the screen 300 is displayed at the upper left of the screen. In the example of FIG. 26, a button for changing the amount of displacement to 5 times (×5) that of the actual waist oscillation is displayed. The amount of displacement (magnification) that can be set and the arrangement of the button are not limited to the example of FIG. 26.

In the state of the upper left screen 300, the button is not pressed. In the state of the upper left screen 300, the subject is displayed at a position right of the following center FC by the absolute value |Dx| of the waist oscillation Dx. In the state of the lower right screen 300, the button is pressed to activate the change in the amount of displacement. In the state of the lower right screen 300, in response to the pressing of the button, the display position of the subject is displayed at a position on the right obtained by enlarging the the waist oscillation Dx by five times (5×|Dx|) the absolute value |Dx| of the waist oscillation Dx. In the state of the lower right screen 300, since the waist oscillation is emphasized, it is easy to recognize the fluctuation in the waist position of the subject during gait.

In the present application example, the amount of displacement of the subject on the screen 300 is changed according to the user's operation. According to the present application example, since the waist oscillation according to the gait of the subject can be highlighted, the gait can be grasped more intuitively.

As described above, the gait information generation device of the present example embodiment includes the acquisition unit, the waist oscillation calculation unit, the following center calculation unit, the gait information generation unit, and the output unit. The acquisition unit acquires waist position information including time-series data of a waist position of the subject in a predetermined gait cycle. The waist oscillation calculation unit calculates the waist oscillation for a plurality of gait phases included in the predetermined gait cycle. The waist oscillation corresponds to a distance between the reference line set for the time-series data of the waist position of the subject and the waist position in each of the plurality of gait phases. The following center calculation unit calculates a following center that follows the subject according to a direction and a magnitude of the waist oscillation. The gait information generation unit generates the gait information according to the waist oscillation calculated with respect to the predetermined gait cycle. The gait information generation unit generates, as the gait information, display information in which the subject is displayed at a position according to a difference between the following center and the waist oscillation in the frame constituting the video indicating the gait state of the subject. The output unit outputs the gait information including the generated display information.

In the present example embodiment, display information is generated in which the subject is displayed at a position corresponding to a difference (waist oscillation) between the following center and the waist position. Therefore, according to the present example embodiment, it is possible to more intuitively grasp the gait of the subject according to the fluctuation in the position of the subject in the frames constituting the video. In the present example embodiment, an example in which the display information in which the subject is displayed at a position corresponding to the waist oscillation is generated is described. The method of the present example embodiment may be applied to generation of display information in which the subject is displayed at a position according to the speed information about the waist oscillation.

### Fourth Example Embodiment

Next, a gait information generation device according to the fourth example embodiment will be described with reference to the drawings. The gait information generation device of the present example embodiment has a configuration in which the first to third gait information generation devices are simplified.

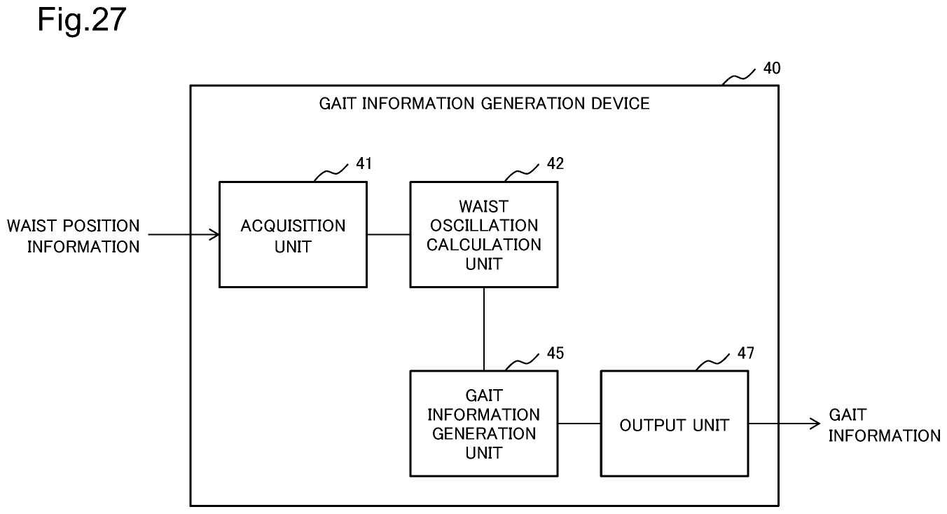

FIG. 27 is a block diagram illustrating an example of a configuration of a gait information generation device 40 according to the present example embodiment. The gait information generation device 40 includes an acquisition unit 41, a waist oscillation calculation unit 42, a gait information generation unit 45, and an output unit 47.

The acquisition unit 41 acquires waist position information including time-series data of a waist position of the subject in a predetermined gait cycle. The waist oscillation calculation unit 42 calculates the waist oscillation corresponding to a distance between a reference line set for the time-series data of the waist position of the subject and the waist position in each of the plurality of gait phases for a plurality of gait phases included in the predetermined gait cycle. The gait information generation unit 45 generates the gait information according to the waist oscillation calculated with respect to the predetermined gait cycle. The output unit 47 outputs the generated gait information.

According to the present example embodiment, the gait information capable of intuitively grasping the gait of the subject is generated by generating the gait information according to the waist oscillation of the subject.

(Hardware)

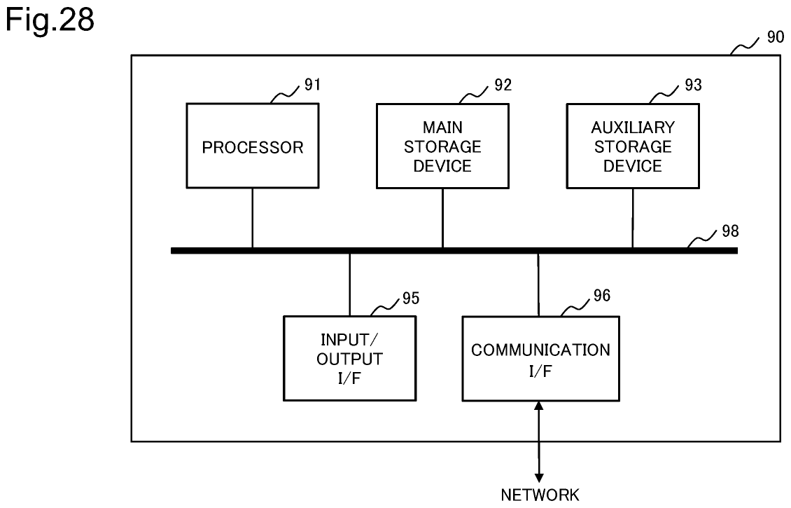

Here, a hardware configuration for executing the processing according to each example embodiment of the present disclosure will be described using an information processing apparatus 90 (computer) of FIG. 28 as an example. The information processing apparatus 90 in FIG. 28 is a configuration example for executing the processing of each example embodiment, and does not limit the scope of the present disclosure.

As illustrated in FIG. 28, the information processing apparatus 90 includes a processor 91, a main storage device 92, an auxiliary storage device 93, an input/output interface 95, and a communication interface 96. In FIG. 28, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the processing of each example embodiment. The processor 91 executes the program developed in the main storage device 92. The processor 91 executes the processing according to each example embodiment by executing the program.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magneto resistive random access memory (MRAM) may be configured/added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing apparatus 90 and a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

Input devices such as a keyboard, a mouse, and a touch panel may be connected to the information processing apparatus 90 as necessary. These input devices are used to input of information and settings. When a touch panel is used as the input device, a screen having a touch panel function serves as an interface. The processor 91 and the input device are connected via the input/output interface 95.

The information processing apparatus 90 may be provided with a display device that displays information. In a case where a display device is provided, the information processing apparatus 90 includes a display control device (not illustrated) for controlling display of the display device. The information processing apparatus 90 and the display device are connected via the input/output interface 95.

The information processing apparatus 90 may be provided with a drive device. The drive device mediates reading of data and a program stored in a recording medium and writing of a processing result of the information processing apparatus 90 to the recording medium between the processor 91 and the recording medium (program recording medium). The information processing apparatus 90 and the drive device are connected via the input/output interface 95.

The above is an example of a hardware configuration for enabling the processing according to each example embodiment of the present invention. The hardware configuration of FIG. 28 is an example of a hardware configuration for executing the processing according to each example embodiment, and does not limit the scope of the present invention. A program for causing a computer to execute processing according to each example embodiment is also included in the scope of the present invention.

A program recording medium recording the program according to each example embodiment is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium is a program recording medium.

The components of the example embodiments may be combined in any manner. The components of the example embodiments may be achieved by software. The components of each example embodiment may be achieved by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A gait information generation device comprising:
at least one memory storing instructions; and
at least one processor connected to the at least one memory and configured to execute the instructions to:
acquire waist position information including time-series data of a waist position of a subject in a predetermined gait cycle;
calculate, for a plurality of gait phases included in the predetermined gait cycle, a waist oscillation corresponding to a distance between a reference line set for the time-series data of the waist position of the subject and the waist position in each of the plurality of gait phases;
generate gait information according to the waist oscillation calculated with respect to the predetermined gait cycle; and
output the generated gait information.

2. The gait information generation device according to claim 1, wherein
the at least one processor is configured to execute the instructions to
acquire the waist position information including time-series data of the waist position of the subject in one gait cycle,
calculate, for the plurality of gait phases included in the one gait cycle, the waist oscillation corresponding to a distance between the reference line set for the time-series data of the waist position of the subject and the waist position in each of the plurality of gait phases, and
generate the gait information including information according to a fluctuation of the waist oscillation calculated with respect to the one gait cycle.

3. The gait information generation device according to claim 1, wherein
the at least one processor is configured to execute the instructions to
acquire the waist position information including time-series data of the waist position of the subject in a plurality of gait cycles,
calculate, for the plurality of gait phases included in the plurality of gait cycles, the waist oscillation corresponding to a distance between the reference line set for the time-series data of the waist position of the subject in each of the plurality of gait cycles and the waist position in each of the plurality of gait phases, and
generate the gait information including information according to a fluctuation of the waist oscillation calculated for each of the plurality of gait cycles.

4. The gait information generation device according to claim 1, wherein
the at least one processor is configured to execute the instructions to
generate, as the gait information, display information in which time-series data of the waist oscillation in the predetermined gait cycle is superimposed on a frame constituting a video indicating a gait state of the subject.

5. The gait information generation device according to claim 4, wherein
the at least one processor is configured to execute the instructions to
generate the display information in which an indicator associated with the gait phase of the subject is superimposed on time-series data of the waist oscillation in the predetermined gait cycle.

6. The gait information generation device according to claim 1, wherein
the at least one processor is configured to execute the instructions to
generate speed information according to a direction and a magnitude of a speed of the waist oscillation,
generate, as the gait information, display information in which an arrow indicating a direction and a magnitude of a speed of the waist oscillation is superimposed on a frame constituting a video indicating a gait state of the subject.

7. The gait information generation device according to claim 1, wherein
the at least one processor is configured to execute the instructions to
calculate a following center that follows the subject according to a direction and a magnitude of the waist oscillation,
generate, as the gait information, display information in which the subject is displayed at a position according to a difference between the following center and the waist oscillation in a frame constituting a video indicating a gait state of the subject.

8. The gait information generation device according to any one of claim 4, wherein
the at least one processor is configured to execute the instructions to
output the display information related to the subject to a terminal device, and
display the display information on a screen of the terminal device.

9. A gait information generation method executed by a computer, the method comprising:
acquiring waist position information including time-series data of a waist position of a subject in a predetermined gait cycle;
calculating, for a plurality of gait phases included in the predetermined gait cycle, waist oscillation corresponding to a distance between a reference line set for the time-series data of the waist position of the subject and the waist position in each of the plurality of gait phases;
generating gait information according to the waist oscillation calculated with respect to the predetermined gait cycle; and
outputting the generated gait information.

10. A non-transitory computer-readable recording medium recording a program for causing a computer to execute:
a process of acquiring waist position information including time-series data of a waist position of a subject in a predetermined gait cycle;
a process of calculating, for a plurality of gait phases included in the predetermined gait cycle, waist oscillation corresponding to a distance between a reference line set for the time-series data of the waist position of the subject and the waist position in each of the plurality of gait phases;
a process of generating gait information according to the waist oscillation calculated with respect to the predetermined gait cycle; and
a process of outputting the generated gait information.
